(19) United States
(12) Patent Application Publication
(10) Pub. No.: US 2023/0204598 A1
(43) Pub. Date: Jun. 29, 2023

(54) MAM-SPECIFIC FLUORESCENCE MARKER AND USE THEREOF

(71) Applicant: POSTECH ACADEMY-INDUSTRY FOUNDATION, Pohang-si (KR)

(72) Inventors: Sang Ki PARK, Pohang-si (KR); Yeong Jun SUH, Gwangju (KR); Bon Seong GOO, Busan (KR)

(21) Appl. No.: 16/316,076

(22) PCT Filed: Nov. 22, 2017
(86) PCT No.: PCT/KR2017/013351

§ 371 Date: Jan. 8, 2019

(30) Foreign Application Priority Data
Nov. 25, 2016 (KR) 10-2016-0157951

(51) Int. Cl. G01N 33/68; G01N 33/58; G01N 21/64

(52) U.S. Cl. CPC G01N 33/6872; G01N 33/582; G01N 21/6428

(57) ABSTRACT

The present discloure relates to a Mitochondria-Associated endoplasmic reticulum Membrane (MAM)-specific targeting bimolecular fluorescence complement system and the use thereof. The system of the present discloure can be utilized under in vivo conditions in contrast to conventional electron microscopy and MAM centrifugation techniques used in order to verify MAM specificity, is far more convenient and accurate than a method of indirectly verifying the specificity of MAM by using respective ER and mitochondria fluorescence markers, and can apply all preexisting genetic techniques for the selection of expression tissues or time, thus gaining the advantage of having high availability.

### TECHNICAL FIELD

The present disclosure relates to a bimolecular fluorescence complement system for mitochondria-associated endoplasmic reticulum membrane (MAM)-specific targeting and a use thereof.

### BACKGROUND ART

In eukaryotic cells, the endoplasmic reticulum (ER) and mitochondria form a micro-contacting part called MAM within an approach distance of 10 to 25 nm, and the MAM has been known to play a critical role in regulating metabolism and calcium signaling by exchanging metabolic substances such as lipids, calcium ions, etc. using such a microstructure.

In addition, recently, in research using experimental techniques such as electron microscopy, live cell fluorescent imaging, etc., the MAM has been continuously reported to be of importance in relation to an immune response, a stress response, regulation of apoptotic signaling, a neurodegenerative disease and a cancer disease (Biochimica et Biophysica Acta 1843 (2014) 2253-2262).

The division of existing cell organelles including the ER, mitochondria, etc. is clearly identified and thus the organelles are easily observed. However, since the MAM corresponds to the contact spot between the ER and mitochondria, there is no clear distinction between the ER and mitochondria, and therefore there is a physical limitation in experimentally observing these organelles. Due to this physical limitation, there is not much information on the MAM compared to its biological importance and continuous research interest in MAM.

That is, while main calcium channel substances such as an inositol 1,4,5-triphosphate receptor (IP3R), voltage dependent anion channel 1 (VDAC1), etc. relating to the MAM have been identified, there still is a difference in MAM structures between research groups, mechanisms of MAM formation and regulation are not identified, and research on the MAM still remains in its early stage due to the lack of MAM-specific experimental techniques.

Meanwhile, bimolecular fluorescence complementation (BiFC) is technology based on an operation principle in which fluorescence can be exhibited only when fragments not exhibiting fluorescence approach very close to each other in a common situation, after a fluorescent material is split into two or more fragments. Generally, a bimolecular fluorescence complement (BiFC) is bound to two or more proteins in use, and the BiFC-bound proteins approach each other to within close proximity to be typically used to determine protein interactions.

### DISCLOSURE

### Technical Problem

Therefore, the inventors performed intensive studies on a MAM-specific fluorescent marker which can prove more simply and clearly MAM specificity using physical properties of the MAM formed by the ER and mitochondria approaching each other to within a distance of 10 to 25 nm, and can be used in vivo, which was impossible by a conventional method, and devised the present disclosure.

Therefore, an object of the present disclosure is to provide a bimolecular fluorescence complement system for MAM-specific targeting, which includes (a) a first fluorescence complementary structure in which a linker peptide and a fluorescent protein sequentially bind to an ER target protein, and (b) a second fluorescence complementary structure in which a linker peptide and a fluorescent protein sequentially bind to a mitochondria target protein.

However, technical problems to be solved in the present disclosure are not limited to the above-described problems, and other problems which are not described herein will be fully understood by those of ordinary skill in the art from the following descriptions.

### Technical Solution

The present disclosure provides a bimolecular fluorescence complement system for MAM-specific targeting, which includes:

(a) a first fluorescence complementary structure in which a linker peptide and a fragment of a fluorescent protein sequentially bind to an ER target protein, and

(b) a second fluorescence complementary structure in which a linker peptide and a fragment of a fluorescent protein sequentially bind to a mitochondria target protein.

In an exemplary embodiment of the present disclosure, the ER target protein is suppressor of actin 1 (SAC1).

In another exemplary embodiment of the present disclosure, a fragment of the SAC1 protein consists of amino acids 521 to 587 of a full-length SAC1 protein.

In still another exemplary embodiment of the present disclosure, the mitochondria target protein is A kinase anchoring protein 1(AKAP1). In yet another exemplary embodiment of the present disclosure, a fragment of the AKAP1 protein consists of amino acids 34 to 63 of a full-length AKAP1 protein.

In yet another exemplary embodiment of the present disclosure, the mitochondria target protein is Mitofusin 1(MFN1).

In yet another exemplary embodiment of the present disclosure, the fragment of the SAC1 protein is encoded by a polynucleotide comprising a base sequence of SEQ ID NO: 1.

In yet another exemplary embodiment of the present disclosure, the fragment of the AKAP1 protein is encoded by a polynucleotide comprising a base sequence of SEQ ID NO: 2.

In yet another exemplary embodiment of the present disclosure, the MFN1 protein is encoded by a polynucleotide comprising a base sequence of SEQ ID NO: 3.

In yet another exemplary embodiment of the present disclosure, the linker peptide is encoded by a polynucleotide consisting of 1 to 8 repeats of a base sequence of SEQ ID NO: 4.

In yet another exemplary embodiment of the present disclosure, the linker peptide is encoded by a polynucleotide consisting of 2 to 4 repeats of the base sequence of SEQ ID NO: 4.

In yet another exemplary embodiment of the present disclosure, the fluorescent protein is a fragment of a Venus protein.

In yet another exemplary embodiment of the present disclosure, the fragment of the Venus protein is encoded by a polynucleotide comprising a base sequence of SEQ ID NO: 5.

In yet another exemplary embodiment of the present discloure, the fragment of the Venus protein is encoded by a polynucleotide comprising a base sequence of SEQ ID NO: 6.

In yet another exemplary embodiment of the present discloure, the fragment of the Venus protein is encoded by a polynucleotide comprising a base sequence of SEQ ID NO: 7.

In addition, the present disclosure provides an expression vector, which includes a polynucleotide encoding the first fluorescence complementary structure.

In addition, the present disclosure provides an expression vector, which includes a polynucleotide encoding the second fluorescence complementary structure.

In addition, the present disclosure provides a MAM-specific fluorescent labeling method using the bimolecular fluorescence complement system for MAM-specific targeting.

### Advantageous Effects

Unlike electron microscopy or MAM fractionation, which has been conventionally used to prove MAM specificity, the system of the present disclosure is able to be applied in vivo, is simpler and more accurate than a method of indirectly proving MAM specificity using ER and mitochondria fluorescent markers, and is highly applicable since all conventional genetic techniques can be applied to select expression tissue or expression time.

In addition, the present disclosure can provide a fluorescent material that can specifically label only the MAM without having an artificial effect on cells by preparing a MAM-specific fluorescent marker only using a minimal targeting gene sequence without specific functionality except targeting the ER or mitochondria, a linker without a biologically acting domain and a fluorescent marker without a side effect, and thus the fluorescent material according to the present disclosure is more safe than any method known conventionally.

### DESCRIPTION OF DRAWINGS

FIG. 1A is a schematic diagram of a recombinant nucleic acid molecule for a MAM-specific fluorescent marker of the present discloure.

FIG. 1B is a schematic diagram illustrating an operation principle of a bimolecular fluorescence complement system for MAM-specific targeting according to the present discloure.

FIG. 2 is a schematic diagram illustrating a difference between the bimolecular fluorescence complement system for MAM-specific targeting according to the present disclosure and a conventional method.

FIG. 3A is recombinant expression vectors for the MAM-specific fluorescent marker of the present discloure.

FIG. 3B is recombinant expression vectors for the MAM-specific fluorescent marker of the present discloure.

FIG. 4 illustrates a result of comparing subcellular localization with conventional ER/mitochondria fluorescent markers by confocal fluorescence microscopy for ER and mitochondrial targeting sequences used in the present disclosure to be normally operated in cells.

FIG. 5A illustrates a result of comparing a MAM-specific fluorescent marker of the present disclosure with conventional ER/mitochondria fluorescent markers by observing intracellular fluorescence patterns to confirm efficiency.

FIG. 5B illustrates a result of observing co-localization coefficients thereof (Mander's Coefficients).

FIG. 5C illustrates a result of fluorescent line analysis thereof.

FIG. 6 illustrates a result of confirming an optimal repeating unit of a linker sequence in the MAM-specific fluorescent marker of the present discloure.

FIG. 7 illustrates a result of verifying MAM specificity of the fluorescent marker of the present disclosure using a drug (MAM suppressor).

### MODES OF THE DISCLOURE

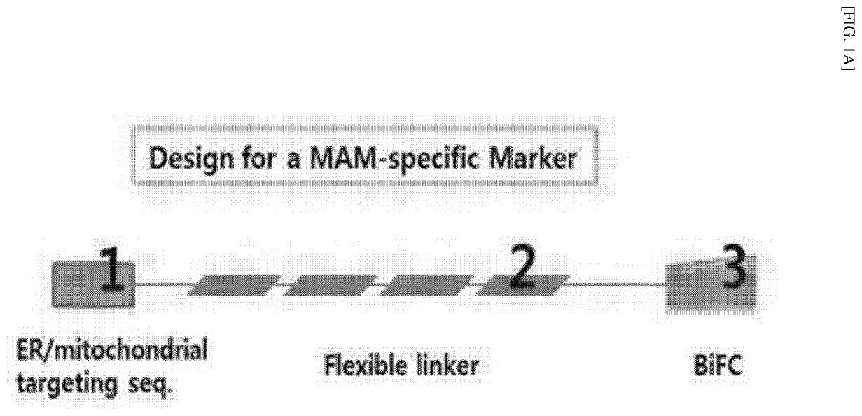

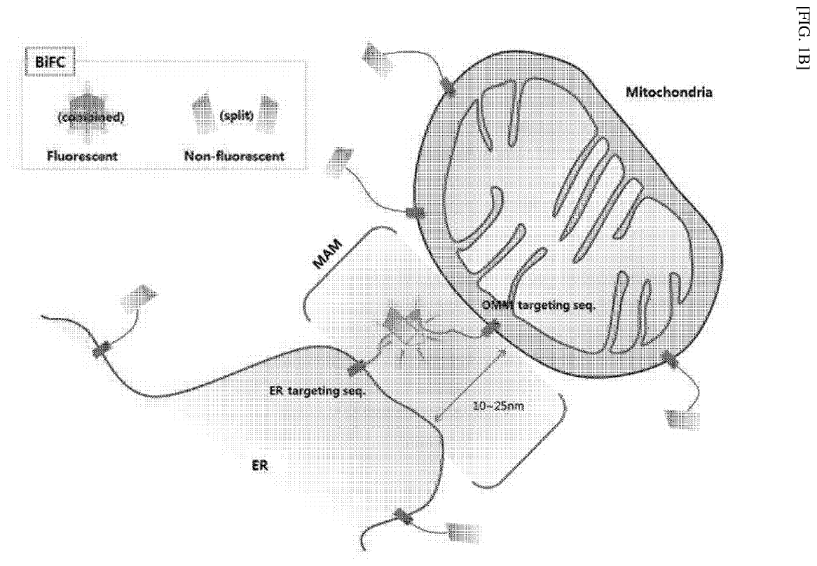

The present disclosure provides a BiFC system for MAM-specific targeting, which includes (a) a first fluorescence complementary structure in which a linker peptide and a fragment of a fluorescent protein sequentially bind to an ER target protein, and (b) a second fluorescence complementary structure in which a linker peptide and a fragment of a fluorescent protein sequentially bind to a mitochondria target protein (see FIGS. 1A and 1B).

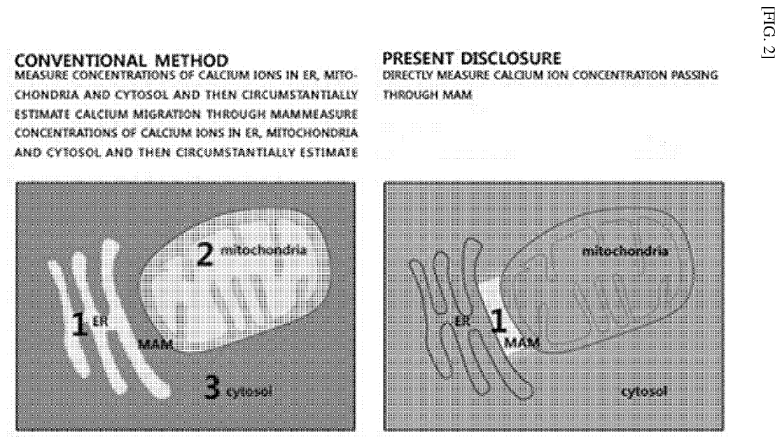

Conventionally, for cell organelles, a specifically targeting fluorescent marker has been widely used as a useful experimental technique, but a marker specifically targeting the MAM, which is a contact spot of the ER and mitochondria, has not be reported yet. For this reason, in most MAM studies, the presence of a specific gene in the MAM was confirmed through methods such as immuno-gold electronic microscopy (IGEM), MAM fractionation, reconstruction of a microscope image of the contact spot of independent ER and mitochondria fluorescent markers (see FIG. 2). However, since all the methods are in vitro experiments which cannot be used in vivo, or methods of observing MAM by indirect methods, these methods may not be independently used to prove a biological phenomenon that occurs in the MAM. Therefore, conventional methods are ineffective because 1) they provide indirect proof and thus have low accuracy, and 2) require much effort and more costs.

However, while there is a report using a fluorescent material that allows linkage between the ER and MAM to develop a more effective MAM-specific method, these studies cannot be considered as techniques suitable for in vivo studies for proving MAM specificity because a MAM structure to be dynamically regulated may be permanently fixed or irreversibly changed by artificial manipulation with a drug.

In the present discloure, a BiFC system is a tool for analyzing fluorescence exhibited when a fluorescent protein to which protein fragment complementation is applied is divided into fragments, each fragment is expressed with two proteins used to investigate their interaction, and then, as the two proteins approach to interact, the two fragments of the fluorescent protein are combined to form a complete fluorescent protein, and in the present discloure, such a BiFC technique was first introduced to implement MAM-specific targeting/fluorescent labeling.

In the present discloure, there is no particular limitation to a ER target protein constituting a first fluorescence complementary structure, as long as it can be specifically targeted in a ER, and a ER target protein may be, for example, calnexin or IP3R(inositol 1,4,5-triphosphate receptor), and preferably, SAC1 (suppressor of actin 1).

Here, a fragment of the SAC1 protein may consist of amino acids 521 to 587 of a full-length SAC1 protein, and may be encoded by a polynucleotide comprising a base sequence of SEQ ID NO: 1 or a base sequence having at least 60%, 70%, 80%, 90% or 95% homology therewith.

In the present discloure, there is no particular limitation to a mitochondria target protein constituting a second fluorescence complementary structure as long as it can be specifically targeted in mitochondria, and the mitochondria target protein may be, for example, TOM20(translocase of outer mitochondrial membrane 20) or VDAC1(voltage dependent anion channel 1), and preferably, AKAP1 (A Kinase Anchoring Protein 1) or MFN1 (Mitofusin 1).

Here, a fragment of the AKAP1 protein may consist of amino acids 34 to 63 of a full-length AKAP1 protein, and may be encoded by a polynucleotide comprising a base sequence of SEQ ID NO: 2 or a base sequence having at least 60%, 70%, 80%, 90% or 95% homology therewith. In addition, the MFN1 protein may be encoded by a polynucleotide comprising a base sequence of SEQ ID NO: 3 or a base sequence having at least 60%, 70%, 80%, 90% or 95% homology therewith.

In the present discloure, there is no limitation to the linker peptide as long as it can link the target protein to the fluorescent protein, and may be encoded by a polynucleotide comprising 1 to 8, and preferably, 2 to 4 repeats of a base sequence of SEQ ID NO: 4 or a base sequence having at least 60%, 70%, 80%, 90% or 95% homology therewith.

At this time, a “sequence homology percent” refers to a degree of identity between any given sequence and a target sequence.

In the present discloure, the fluorescent protein can be used to measure fluorescence after being introduced into cells as a fluorescent protein that can be used in BiFC assay for analyzing protein-protein interaction, and dimerization or oligomerization in cells, and a type of a fluorescent protein is not particularly limited. Preferably, the fluorescent protein may be selected from a Venus protein, a green fluorescent protein (GFP), a yellow fluorescent protein (YFP), a red fluorescent protein (RFP), a cyan fluorescent protein (CFP), a blue fluorescent protein (BFP), ECFP, TagCFP, DsRed, and mCherry, and the fluorescent protein may be designed in various sizes according to the type, characteristic, stability and fluorescence intensity of the protein.

More preferably, the fluorescent protein is a Venus protein fragment encoded by a polynucleotide comprising a base sequence of SEQ ID NO: 5, 6 or 7. The Venus protein is a fluorescent protein containing F46L, F64L, S65G, V68L, S72A, M153T, V163A, S175G and T203Y mutants in enhanced GFP.

In addition, the present disclosure provides a recombinant expression vector that expresses a protein in which an ER or mitochondria target protein is fused with a fluorescent protein via a linker peptide.

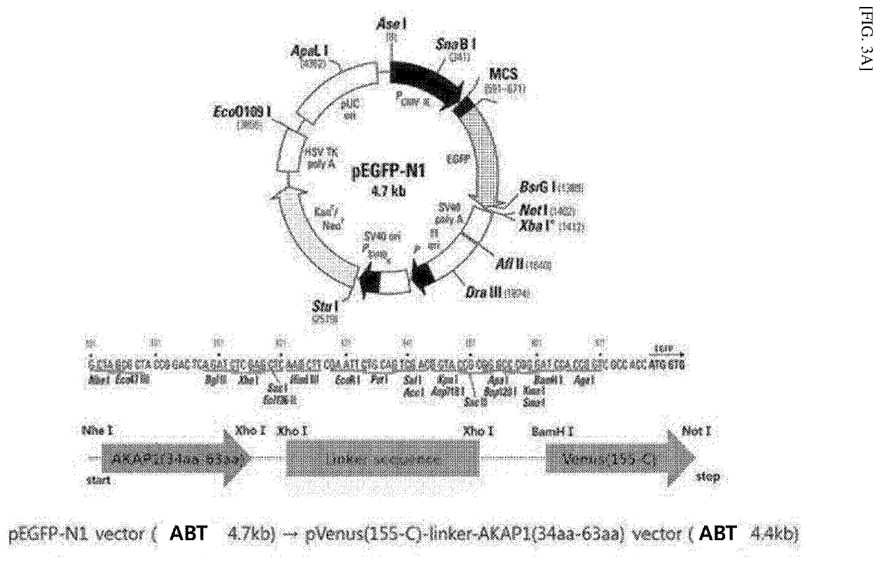

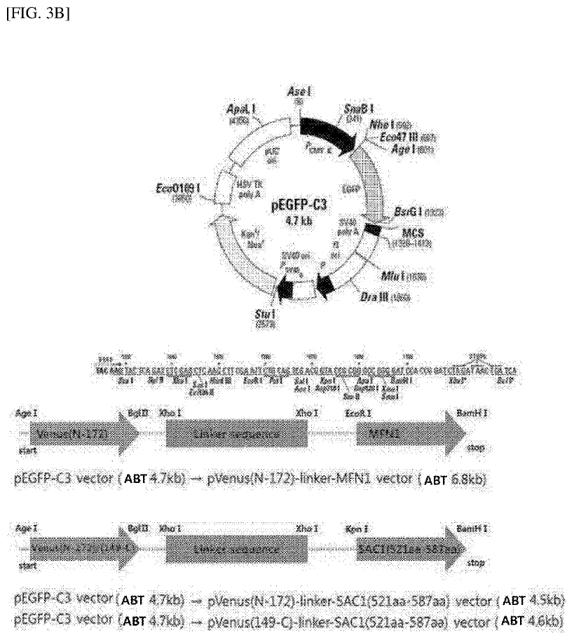

The “vector” used herein may be a random material that can deliver and express a nucleic acid molecule in host cells or a test specimen. Therefore, a vector may be a replicon, for example, a plasmid, a phage or a cosmid, into which a PCR product or a random nucleic acid fragment introduced into cells and integrated into a cell genome may be inserted. Generally, a vector may be replicated when combined with a suitable regulatory element. The backbone of the vector suitable for being used in the present disclosure may be prepared to be expressed by a promoter exhibiting high expression efficiency in mammalian cells, and include, for example, a CMV promoter. Preferably, pEGFP-N1 and pEGFP-C3 vectors shown in FIGS. 3A and 3B may be used as backbones.

In the present discloure, there is no limitation to a method of preparing a fusion gene by cloning a desired gene in the vector backbone, and for example, blunt-ended termini or stagger-ended termini for ligation, digestion using a restriction enzyme for providing a suitable terminus, interlocking of cohesive ends as needed, treatment of an alkaline phosphatase to avoid undesired bonding, and enzymatic ligation may be used.

In the present discloure, since the target protein-linker peptide may be fused with the N terminus or C terminus region of a fluorescent protein through peptide bonding, thereby forming a fusion protein expressed as a polypeptide, and since a linker peptide may be bonded to both of the C- and N-termini of the fluorescent protein, it may be expressed in the form of a (fluorescent protein terminal region)-linker or linker-(fluorescent protein terminal region).

In the present discloure, as a recombinant expression vector of the present disclosure is transfected into cells, the cells are cultured to express a protein therein, and fluorescence from the cells was measured, it can be confirmed that a specific location in cells is targeted, and a protein-protein interaction can be exactly analyzed. Here, fluorescence may be measured using a fluorescent microscope or confocal microscope.

In addition, according to the present discloure, a MAM-specific fluorescent labeling method may be provided using the bimolecular fluorescence complement system for MAM-specific targeting.

Hereinafter, to help in understanding the present discloure, exemplary examples will be suggested. However, the following examples are merely provided to more easily understand the present discloure, and not to limit the present discloure.

### EXAMPLES

### Example 1. Preparation of Mitochondrial Targeting Recombinant Nucleic Acid Molecule Using AKAP1

### 1-1. Insertion of Mitochondrial Targeting Sequence

Based on a pEGFP-N1 vector, a mitochondrial targeting sequence (the gene sequence of 90 base pairs corresponding to the sequence of amino acids 34 to 63) of mouse Akap1 gene (A kinase (PRKA) anchor protein 1, Mus musculus, Gene ID: 11640) was amplified, and inserted as a mitochondrial targeting sequence of a recombinant gene. To this end, a mouse cDNA library was used as a template, and PCR was performed using primers having the following sequences.

AKAP1-(34 aa-63 aa) forward primer:
5’-ctagctagccaccatggcaatccagttgcgttcg-3’
AKAP1-(34 aa-63 aa) reverse primer:
5’-ccgctcgagttttttacgagagaaaaaccaccaccagcc-3’

Amplified DNA was treated with Nhe I and Xho I restriction enzymes, and inserted into a pEGFP-N1 vector cleaved with the Nhe I and Xho I restriction enzymes using a T4 ligase, thereby manufacturing a pEGFP-N1-AKAP1(34aa-63aa) vector.

### 1-2. Replacement of Fluorescent Protein Gene

PCR was performed on a gene encoding Venus, which is a fluorescent protein containing F46L, F64L, S65G, V68L, S72A, M153T, V163A, S175G and T203Y mutants in enhanced GFP as a template using the following primers.

Venus155-N1 forward primer:
5’-cgcggatcccaccatgaagcagaagaacggcatcaag-3’
Venus155-N1 reverse primer:
5′-aaatatgcggccgctttacttgtacagctcgtccatgc-3’

Amplified DNA was treated with BamH I and Not I restriction enzymes, and inserted into the pEGFP-N1-AKAP1(34aa-63aa) vector from which an EGFP gene was deleted using the BamH I and Not I restriction enzymes using a T4 ligase, thereby manufacturing a pVenus(155-C)-N1-AKAP1(34aa-63aa) vector in which an EGFP gene is substituted with a BiFC gene.

### 1-3. Insertion of Linker Sequence

In this example, the following linker sequence of 60 bp was used once or repeatedly as needed.

linker sequence:

5’-gacccaaccaggtcagcgaattctggagcaggagcaggagcaggag
caatactctcccgt-3’

Specifically, a linker sequence of the following sequence was synthesized, the synthesized oligo DNA was treated with Xho I and Sal I restriction enzymes and inserted into the pVenus(155-C)-N1-AKAP1(34aa-63aa) vector treated with an Xho I restriction enzyme using a T4 ligase, thereby manufacturing a pVenus(155-C)-linker-AKAP1(34aa-63aa) vector (see FIG. 3A).

linker oligo DNA: 5′-ccgctcgag

(gacccaaccaggtcagcgaattctggagcaggagcaggagcaggagca
atactctcccgt)n gtcgac-3’

### Example 2. Manufacture of Mitochondrial Targeting Recombinant Nucleic Acid Molecule Using MFN1

### 2-1. Insertion of Mitochondrial Targeting Sequence

Based on a pEGFP-C3 vector, another mitochondrial targeting vector was manufactured by amplifying a mouse Mfnl gene (mitofusin 1, Mus musculus, Gene ID: 67414).

To this end, PCR was performed using a mouse cDNA library as a template and primers of the following sequences.

MFN1 forward primer:
5’-ccggaattctggcagaaacggtatctccactgaag-3’
MFN1 reverse primer:
5’-cgcggatccttaggattctccactgctcggg-3’

Amplified DNA was treated with EcoR I and BamH I restriction enzymes, and inserted into a pEGFP-C3 vector cleaved with EcoR I and BamH I restriction enzymes using a T4 ligase, thereby manufacturing a pEGFP-C3-MFN1 vector.

### 2-2. Replacement of Fluorescent Protein Gene

PCR was performed using a Venus gene as a template and primers as follows.

VenusN172-C3 forward primer:
5’-gggaccggtgccaccatggtgagcaagggcgag-3’
VenusN172-C3 reverse primer:
5’-ggaagatctgactcgatgttgtggcggatc-3’

Amplified DNA was treated with Age I and Bgl II restriction enzymes, and inserted into a pEGFP-C3-MFN1 vector from which an EGFP gene was deleted with the Age I and Bgl II restriction enzymes using a T4 ligase, thereby manufacturing a pVenus(N-172)-C3-MFN1 vector in which an EGFP gene is substituted with a BiFC gene.

### 2-3. Insertion of Linker Sequence

As described in Example 1-3, a linker sequence (60 bp) was used once or repeatedly as needed, and oligo DNA synthesized in the same manner as described in Example 1-3 was treated with Xho I and Sal I restriction enzymes and inserted into the pVenus(N-172)-C3-MFN1 vector treated with an Xho I restriction enzyme using a T4 ligase, thereby manufacturing a pVenus(N-172)-linker-MFN1 vector (see FIG. 3B).

### Example 3. Manufacture of ER Targeting Recombinant Nucleic Acid Molecule Using SAC1

### 3-1. Insertion of ER Targeting Sequence

Based on a pEGFP-C3 vector, an ER targeting sequence (the gene sequence of 204 base pairs corresponding to the sequence of amino acids 521 to 587) of a mouse Sac 1 gene (SAC1; suppressor of actin mutations 1-like (yeast), Mus musculus, Gene ID: 83493) was amplified, and inserted as an ER targeting sequence of a recombinant gene.

To this end, a mouse cDNA library was used as a template, and PCR was performed using primers of the following sequences.

SAC1-(521 aa-587 aa) forward primer:
5’-cggggtaccgttcctggcgttgcctatcatc-3’
SAC1-(521 aa-587 aa) reverse primer:
5’-cgcggatcctcagtctatcttttctttctggaccag-3’

Amplified DNA was treated with Kpn I and BamH I restriction enzymes, and inserted into a pEGFP-C3 vector cleaved with the Kpn I and BamH I restriction enzymes using a T4 ligase, thereby manufacturing a pEGFP-C3-SAC1(521aa-587aa) vector.

### 3-2. Replacement of Fluorescent Protein Gene

PCR was performed using a Venus gene as a template and the following primers.

Venus149C-C3 forward primer:
5’-gggaccggtgccaccatgaacgtctatatcaccgccgac-3’
Venus149C-C3 reverse primer:
5’-ggaagatctgacttgtacagctcgtccatgcc-3’

Amplified DNA was treated with Age I and Bgl II restriction enzymes, and inserted into a pEGFP-C3-SAC1(521aa-587aa) vector from which an EGFP gene was deleted with the Age I and Bgl II restriction enzymes using a T4 ligase, thereby manufacturing a pVenus(149-C)-C3-SAC1(521aa-587aa) vector in which an EGFP gene is substituted with a BiFC gene.

PCR was performed using the Venus gene as a template and the following primers.

VenusN172-C3 forward primer:
5’-gggaccggtgccaccatggtgagcaagggcgag-3’
VenusN172-C3 reverse primer:
5’-ggaagatctgactcgatgttgtggcggatc-3’

Amplified DNA was treated with Age I and Bgl II restriction enzymes, and inserted into a pEGFP-C3-SAC1(521aa-587aa) vector from which an EGFP gene was deleted with Age I and BglII restriction enzymes using a T4 ligase, thereby manufacturing a pVenus(N-172)-C3-SAC1(521aa-587aa) vector in which an EGFP gene is substituted with a BiFC gene.

### 3-3. Insertion of Linker Sequence

As described in Example 1-3, a linker sequence (60 bp) was used once or repeatedly as needed, and oligo DNA synthesized in the same manner as described in Example 1-3 was treated with Xho I and Sal I restriction enzymes and inserted into a pVenus(149-C)-C3-SAC1(521aa-587aa) vector and a pVenus(N-172)-C3-SAC1(521aa-587aa) vector, which were treated with an Xhol restriction enzyme, using a T4 ligase, thereby manufacturing a pVenus(149-C)-linker-SAC1(521aa-587 aa) vector and a pVenus(N-172)-linker-SAC1(521aa-587aa) vector (see FIG. 3B).

### Example 4. Verification of ER/Mitochondrial Targeting

The following experiments were performed to confirm whether the ER/mitochondrial targeting sequences manufactured in Examples 1 to 3 are actually effective in ER/mitochondria-specific targeting in cells.

### 4-1. Transfection of Recombinant Vector

HEK293 cells were cultured on a cover glass coated with poly-D-lysine for 12 hours, and then the pEGFP-N1-AKAP1(34aa-63aa) vector (mitochondrial targeting), the pEGFP-C3-MFN1 vector (mitochondrial targeting) and the pEGFP-C3-SAC1(521aa-587aa) vector (ER targeting), manufactured in Examples 1-1, 2-1 and 3-1, were transfected into HEK293 cells cultured together with a mitochondria fluorescent marker gene vector and an ER fluorescent marker gene vector.

Here, the fluorescent marker gene used herein employed a mitochondria fluorescent marker in which the N-terminus 29aa of human COXVIII was linked to an mCherry gene and an ER fluorescent marker (Plasmid #38770) substance registered to Addgene, and a Lipofectamine 2000 reagent produced by Invitrogen as a transfection reagent was used according to the manufacturer's protocol.

### 4-2. Preparation and Observation of Microscope Sample

HEK293 cells transfected in Example 4-1 were incubated in DMEM containing a 10% fetal bovine serum (FBS) for 24 hours under conditions of 37° C. and 5% CO₂. Afterward, the culture solution was washed with PBS, and then cells were fixed by treatment with a 4% para-formaldehyde cell fixing solution for 10 minutes. The cell fixing solution was sufficiently washed with PBS, and a cover glass was immobilized on a slide glass using a mounting solution, thereby manufacturing a microscope sample.

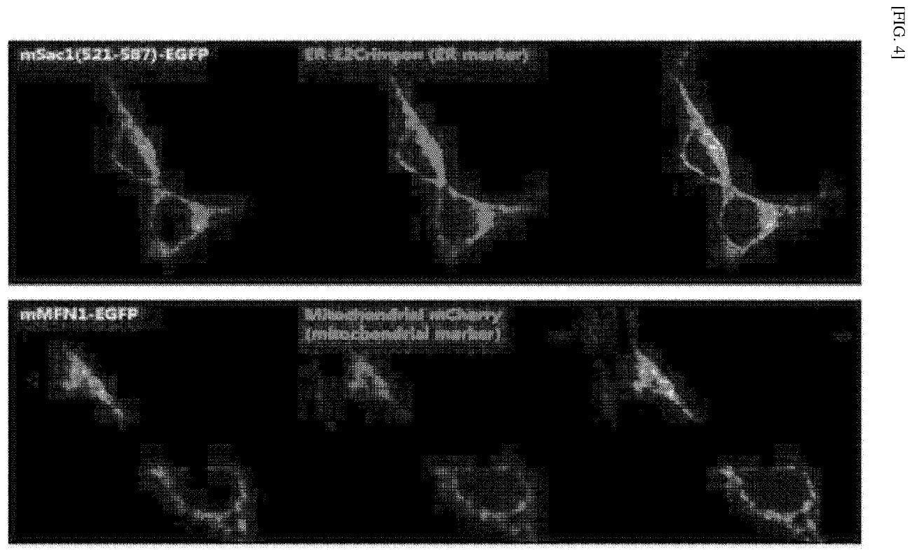

Afterward, as a result of analyzing a colocalization level by observing an intracellular fluorescence pattern using a fluorescent microscope, as shown in FIG. 4, by comparing fluorescence patterns of the ER labeling fluorescent material and the mitochondria labeling fluorescent material, it was experimentally proven that SAC1(521aa-587aa) and MFN1 sequences are effective as an ER targeting sequence and a mitochondrial targeting sequence, respectively.

### Example 5. Verification of MAM Targeting

### 5-1. Preparation and Observation of Microscope Sample

To verify MAM targeting of the biomolecular MAM-specific fluorescent markers manufactured in the present discloure, the pVenus(155-C)-linker-AKAP1(34aa-63aa) vector, the pVenus(N-172)-linker-MFN1 vector, the pVenus (149-C)-linker-SAC1(521 aa-587aa) vector and the pVenus(N-172)-linker-SAC1(521aa-587aa) vector manufactured in Examples 1-3, 2-3 and 3-3 were transfected into HEK293 cells along with a mitochondria fluorescent marker gene vector and an ER fluorescent marker gene vector in the same manner as described in Example 4, thereby manufacturing microscope samples.

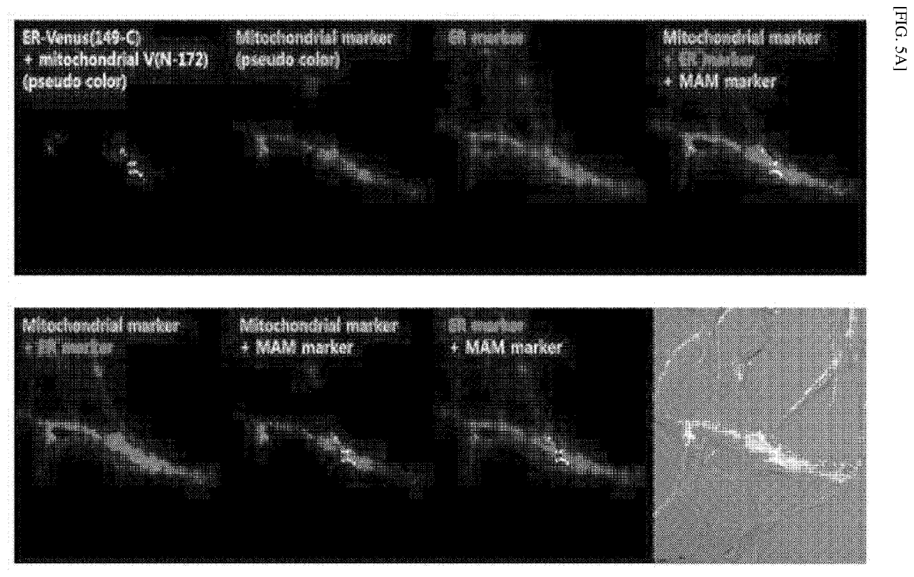

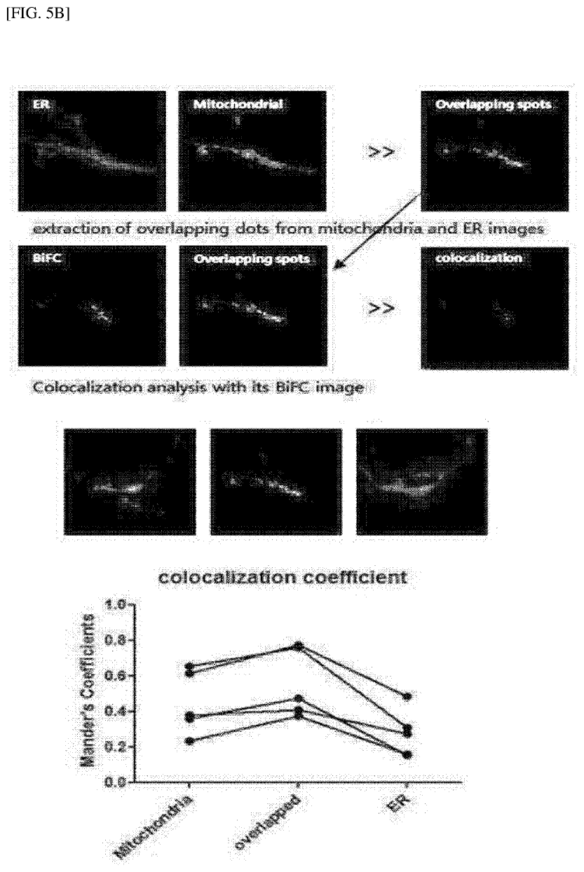

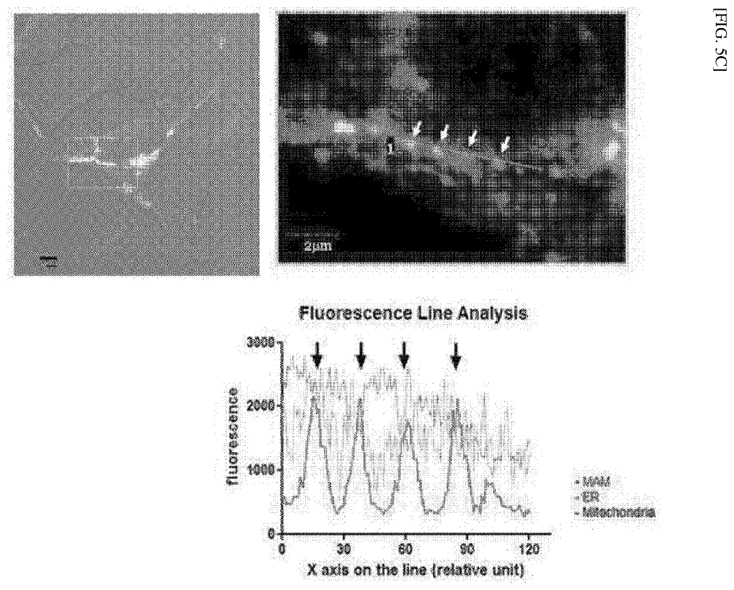

Afterward, as a result of observing an intracellular fluorescence pattern using a fluorescent microscope, as shown in FIG. 5A, it was confirmed that the MAM-specific bimolecular fluorescent markers using these targeting sequences selectively label a contact spot of the ER and mitochondria (MAM) in cells.

### 5-2. Analysis of MAM Targeting

To analyze a MAM targeting level of the bimolecular MAM-specific fluorescent marker manufactured in the present discloure, fluorescent images obtained using the microscope sample obtained in Example 5-1 were analyzed using a universal image analysis program ImageJ, which is distributed by NIH.

First, co-localization coefficients (Mander's Coefficients) with a fluorescence pattern of the bimolecular MAM-specific fluorescent marker were analyzed by extracting overlapping spots between fluorescence of the ER-labeling fluorescent material and the mitochondria-labeling fluorescent material, which label an ER substrate and a mitochondria substrate, respectively, by a method used in conventional MAM studies.

As a result, as shown in FIG. 5B, in the case of the bimolecular MAM-specific fluorescent marker of the present discloure, it was confirmed that, in all monitored cells, fluorescent labeling of each of mitochondria and the ER was more highly overlapped at sites in which mitochondria and ER fluorescence overlapped.

In addition, to analyze patterns of a MAM-specific fluorescent marker, an ER fluorescent marker and a mitochondria fluorescent marker in a section in which a MAM-specific fluorescent marker signal was shown, line analysis was performed.

As a result, as shown in FIG. 5C, it was confirmed that the MAM present at the boundary (the part in which the solid line is overlapped in the graph, represented by arrows) between mitochondria (blue fluorescence) and the ER (red fluorescence) is very exactly represented by the MAM-specific bimolecular fluorescent marker of the present discloure.

### Example 6. Confirmation of Optimal Repeating Unit of Linker Sequence

To confirm the optimal repeat number of a linker sequence, a linker sequence (SEQ ID NO: 4) of 60 bp was repeatedly inserted as a unit to manufacture a linker having various repeat numbers, and then a MAM-specific fluorescence pattern was observed.

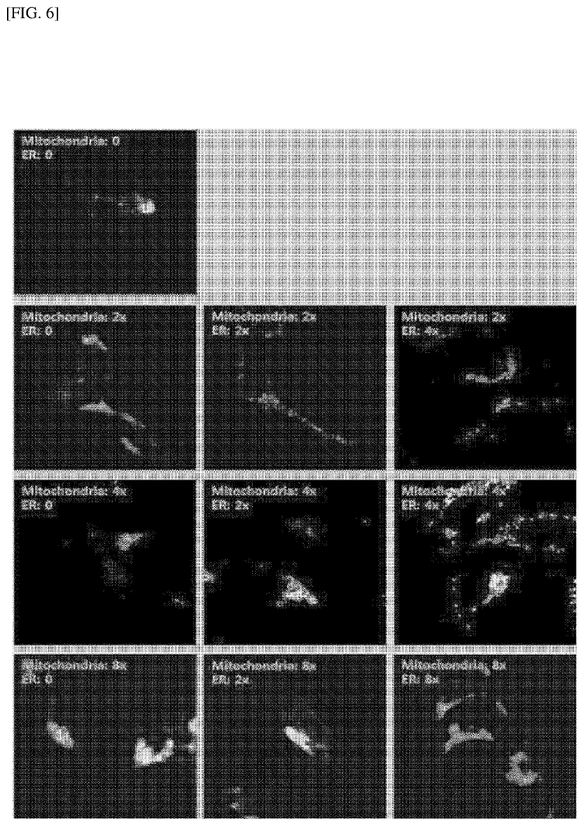

As a result, as shown in FIG. 6, from the combination of a vector (pVenus(155-C)-2*linker-AKAP1(34aa-63aa)) and a vector (pVenus(149-C)-2*linker-SAC1(521aa-587aa)) in which two linker units are inserted into a mitochondrial targeting sequence and an ER targeting sequence among linker sequence-inserted gene substances with various lengths, respectively, the most-specific fluorescent pattern to MAM was able to be confirmed.

In other words, when the linker sequence was very short, the cells exhibited an abnormal type of fluorescence, and when the linker sequence was very long, a phenomenon in which MAM specificity was reduced in the form of covering the periphery of the outer membrane of mitochondria with fluorescence was confirmed.

### Example 7. Verification of MAM Specificity of Fluorescent Marker of the Present Disclosureusing Drug

The MAM-specific bimolecular fluorescent marker was transfected into HEK293 cells in the same manner as in Example 5-1, 150 μM of methyl-β-cyclodextrin known as a MAM suppressor was added to the cell culture solution, and the cells were incubated for 3 or 24 hours under cell culture conditions, thereby manufacturing a microscope sample, and then the microscope sample was observed using a fluorescence microscope.

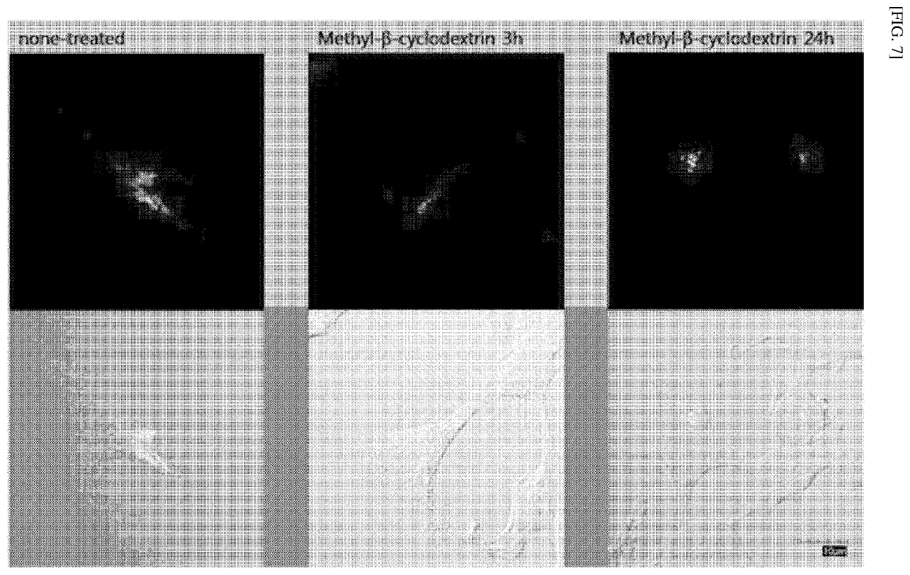

As a result, as shown in FIG. 7, when a MAM structure is scattered and suppressed through treatment of methyl-β-cyclodextrin, it was confirmed that a fluorescence level of the MAM-specific fluorescent marker of the present disclosure was significantly reduced. To this end, it was proven that the MAM-specific fluorescent marker manufactured in the present disclosure is effective in labeling a previously-formed MAM structure, and measuring a change in MAM structure, which dynamically occurs in cells.

It should be understood by those of ordinary skill in the art that the above description of the present disclosure is exemplary, and the exemplary embodiments disclosed herein can be easily modified into other specific forms without departing from the technical spirit or essential features of the present discloure. Therefore, the exemplary embodiments described above should be interpreted as illustrative and not limited in any aspect.

### INDUSTRIAL APPLICABILITY

The bimolecular fluorescence complement system for MAM-specific targeting according to the present disclosure can only label the MAM exactly and safely in cells, and thus can be widely applied in research of MAM functions, various intracellular reactions related thereto and research of related diseases such as degenerative brain diseases or cancer.

1. A bimolecular fluorescence complement system for mitochondria-associated endoplasmic reticulum membrane(MAM)-specific targeting, comprising:
(a) a first fluorescence complementary structure in which a linker peptide and a fragment of a fluorescent protein are sequentially bound to a fragment of an endoplasmic reticulum(ER) target protein; and
(b) a second fluorescence complementary structure in which a linker peptide and a fragment of a fluorescent protein are sequentially bound to a fragment of a mitochondria target protein.

2. The system according to claim 1, wherein the ER target protein is suppressor of actin 1(SAC1).

3. The system according to claim 2, wherein a fragment of the SAC1 protein consists of amino acids 521 to 587 of a full-length SAC1 protein.

4. The system according to claim 1, wherein the mitochondria target protein is A Kinase Anchoring Protein 1(AKAP1).

5. The system according to claim 4, wherein a fragment of the AKAP1 protein consists of amino acids 34 to 63 of a full-length AKAP1 protein.

6. The system according to claim 1, wherein the mitochondria target protein is Mitofusin 1(MFN1).

7. The system according to claim 3, wherein the fragment of the SAC1 protein is encoded by a polynucleotide comprising a base sequence of SEQ ID NO: 1.

8. The system according to claim 5, wherein the fragment of the AKAP1 protein is encoded by a polynucleotide comprising a base sequence of SEQ ID NO: 2.

9. The system according to claim 6, wherein the MFN1 protein is encoded by a polynucleotide comprising a base sequence of SEQ ID NO: 3.

10. The system according to claim 1, wherein the linker peptide is encoded by a polynucleotide having 1 to 8 repeats of a base sequence of SEQ ID NO: 4.

11. The system according to claim 10, wherein the linker peptide is encoded by a polynucleotide having 2 to 4 repeats of the base sequence of SEQ ID NO: 4.

12. The system according to claim 1, wherein the fluorescent protein is a fragment of a Venus protein.

13. The system according to claim 12, wherein the fragment of the Venus protein is encoded by a polynucleotide comprising a base sequence of SEQ ID NO: 5.

14. The system according to claim 12, wherein the fragment of the Venus protein is encoded by a polynucleotide comprising a base sequence of SEQ ID NO: 6.

15. The system according to claim 12, wherein the fragment of the Venus protein is encoded by a polynucleotide comprising a base sequence of SEQ ID NO: 7.

16. An expression vector comprising:
a polynucleotide encoding the first fluorescence complementary structure of claim 1.

17. An expression vector comprising:
a polynucleotide encoding the second fluorescence complementary structure of claim 1.

18. A mitochondria-associated endoplasmic reticulum membrane(MAM)-specific fluorescent labeling method using the system of claim 1.


SEQUENCE LISTING (7 sequences)

SEQ ID NO: 1 - DNA, 204 bp, Mus musculus
  Sac1(521aa-587aa)
ttcctggcgttgcctatcatcatggttgttgccttttcaatgtgcatcatctgtttgcttatggctggtgacacttggacagaaacactggcatatgtcctcttctggggagttgcaagcattggaacattttttattattctttacaatggcaaagattttgttgatgctcccagactggtccagaaagaaaagatagactga

SEQ ID NO: 2 - DNA, 90 bp, Mus musculus
  AKAP1(34aa-63aa)
atggcaatccagttgcgttcgctcttccccttggcgttgcccggaatgctggccctccttggctggtggtggtttttctctcgtaaaaaa

SEQ ID NO: 3 - DNA, 2226 bp, Mus musculus
  MFN1
atggcagaaacggtatctccactgaagcactttgtgctggcaaagaaagccatcactgcaatcttcggccagttactggagtttgttactgagggctcacattttgttgaagcaacatacaggaatccagaacttgatcgaatagcatccgaggatgatctggtggaaatacagggctacagaaacaagcttgctgtcattggggaggtgctgtctcggagacatatgaaggtggcattttttggcaggacaagtagtggcaagagctctgtcatcaatgcaatgctgtgggataaagtcctccccagcgggattggtcacacaaccaactgcttcctgagtgtcgaggggaccgatggagataaagcctaccttatgaccgaagggtcagatgaaaagaaaagtgtgaagactgttaatcagctggcccatgccctccatatggataaagacttgaaagctggctgtcttgtgcatgtattttggcccaaagcaaaatgtgccctcttgagagatgacctggttttagtagacagcccaggtacagatgtcaccacagagctggatatctggattgataagttttgccttgatgctgatgtctttgttttggttgcaaactcggaatcaacactgatgaacacggagaaacattttttccataaggtgaatgagcggctctccaagcccaacatcttcattctgaataaccgttgggatgcttctgcttcggagccggagtacatggaggatgtgcgcagacagcacatggagagatgtcttcacttcttggtagaagagctcaaggttgtaagtccgtcggaagctcggaatcggatcttttttgtttcagccaaggaagttctcaactccagaaagcataaagctcaggggatgccagaaggtggtggggcacttgcagaaggatttcaagcaagattacaggagtttcaaaattttgaacaaacttttgaggagtgtatctcgcagtcagcagtgaaaacaaagtttgaacagcacactatcagagctaaacagatactagacactgtgaaaaacatactggactcagtaaacgtggcagcagcagagaagagggtttattcaatggaagagagggaagaccaaatcgatagactggactttatccgaaaccagatgaaccttttaacactggatgttaagaagaagatcaaggaggtcacggaggaggtggcaaacaaggtttcttgtgcaatgacagatgaaatttgtcgactatctgttttggttgatgagttttgttctgagtttcatcctacccccagtgtactgaaagtgtataagagtgagttaaataagcacatagaagatggcatgggaagaaatttggctgatcggtgtaccaatgaagtcaatgcctccattcttcaatctcagcaagaaatcatcgaaaacttgaagccactacttccagctggtatacagaataaacttcatacattaatcccttgcaaaaagtttgacctcagctatgatctcaattgccacaagctgtgttcggattttcaagaggacattgtgtttcggttttccctgggctggtcttcccttgtacatcgattcctgggttccacaaatgcacagagggtgctgctcgggctgtcagagcccatctttcaggtccctagatctttagcttcaactcctactgctccttctaacccagcagccccggataatgcagcccaggaggagctcatgatcaccctgatcacaggattggcgtccctcacgtcgagaacctccatgggcatcatcgttgttgggggcgtgatttggaaaacagtgggctggaaactaatctctgtcaccttaagtatgtacggagctctgtacctttatgagaggctgacgtggacgacccgtgcgaaagagagagcgtttaagcagcagtttgtaaactatgcaaccgagaagctgcagatgattgtgagcttcaccagtgcaaactgcagccaccaagtacagcaagaaatggccactacttttgctcgactgtgccaacaagttgatgttactcagaaacatctggaagaggaaattgcaagattatccaaagagatagaccaactggagaaaatacagaacaactcaaagctcttaagaaataaagctgttcaacttgaaagtgagctggagaatttttcgaagcagtttctacacccgagcagtggagaatcctaa

SEQ ID NO: 4 - DNA, 60 bp, Artificial Sequence
  linker
gacccaaccaggtcagcgaattctggagcaggagcaggagcaggagcaatactctcccgt

SEQ ID NO: 5 - DNA, 516 bp, Mus musculus
  Venus(N-172)
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgag

SEQ ID NO: 6 - DNA, 273 bp, Mus musculus
  Venus(149-C)
aacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 7 - DNA, 252 bp, Mus musculus
  Venus(155-C)
aagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa